(19) United States
(12) Patent Application Publication
(10) Pub. No.: US 2006/0024836 A1
(43) Pub. Date: Feb. 2, 2006

(54) Quantitative analysis of nitrogen oxides occluded in heterogeneous catalysis

(76) Inventors: Jeong Ki Kil, Anyang-si (KR); In Sik Nam, Pohang-si (KR); Joo-Hyoung Park, Busan (KR); Sang Jun Park, Gimhae-si (KR)

Correspondence Address: MORGAN, LEWIS & BOCKIUS LLP (SF), 2 PALO ALTO SQUARE, 3000 El Camino Real, Suite 700, PALO ALTO, CA, 94306, US

(21) Appl. No.: 11/188,937

(22) Filed: Jul. 25, 2005

(30) Foreign Application Priority Data
Jul. 23, 2004 (KR) 10-2004-0057606

(51) Int. Cl. G01N 33/00

(52) U.S. Cl. 436/113; 422/61

(57) ABSTRACT

A method of performing quantitative analysis of nitrogen oxides occluded in heterogeneous catalysts, comprising the following steps, each performed in parallel for multiple samples: adsorption of nitrogen oxides by multiple samples using a batch adsorption apparatus, extraction of nitrogen oxides from the occluded samples using a solvent, reduction of nitrogen oxides to nitrite ions contained in the extract using hydrazine, and detection and quantification of nitrogen oxides by colorization using a modified Griess reagent. The method of the invention requires less time and expense as compared to the conventional method, wherein the process of serial adsorption, purge, desorption, and quantification is performed repeatedly to quantify the amount of nitrogen oxides adsorbed onto multiple samples. This method enables the efficient and reliable evaluation of nitrogen oxide adsorbing catalysts, which are commonly employed in automobiles for post-treatment of exhaust gases.

### CROSS-REFERENCE TO RELATED APPLICATION

This application claims priority to Korean Patent Application No. 10-2004-0057606, filed on Jul. 23, 2004, the disclosure of which is hereby incorporated by reference in its entirety.

### BACKGROUND OF THE INVENTION

This invention relates to a method of performing quantitative analysis of nitrogen oxides occluded in heterogeneous catalysis.

In general, nitrogen oxides (NOₓ) are chemicals derived from the combustion of petroleum or coal. In addition to nitrogen oxides (NOₓ), exhaust gases produced by automobiles also contain hydrocarbons (HC) and carbon monoxide (CO). When exposed to the air, nitrogen monoxide (NO) generated from combustion oxidizes into nitrogen dioxide (NO₂). Nitrogen oxides can cause respiratory diseases, such as emphysema and bronchial diseases, and also generate ozone, a major cause of global warming and acid rain, through photochemical reactions induced by strong sunlight.

Many countries are now establishing strict regulations to reduce air pollution caused by nitrogen oxide and its accompanying harm to the environment and public health. For instance, Europe declared the EURO III, IV and V. The United States passed the Clean Air Act requiring nitrogen oxide emissions from gasoline-fueled automobiles be reduced to ¼ the level in 1990 by the year 2003. Japan's Air Pollution Prevention Act is amongst the more stringent regulations by barring the sale of automobiles that emits over a certain level of nitrogen oxides, soot, particulates.

As regulations on exhaust gases of automobiles became more and more stringent, it became ever more difficult for the automobile industry to satisfy these heightened requirements merely by virtue of improving engines. Thus, the industry began to take a twofold approach in minimizing nitrogen oxide emissions by advancing methods in post-treatment of exhaust gases and improving of engines. A catalytic converter was installed inside the exhaust pipe in order to reduce the nitrogen oxides contained in engine exhaust gas emissions. In the mid-1980s, a commonly employed technique for this purpose was the three-way catalyst. The three-way catalyst commonly uses platinum group metals, such as platinum, palladium, and rhodium. If the emission temperature is kept sufficiently high, nitrogen oxide acts as an oxidant and carbon monoxide and hydrocarbons act as reductants in a chemical reaction resulting in the simultaneous removal of three pollutants from the exhaust gas: NOₓ, CO, and HC. The reaction would then yield water, carbon dioxide, and nitrogen gas. Despite the promise of 3-way catalyst as an epochal low-emission technique, it does not function optimally in the exhaust stream from lean burn engine, developed to improve fuel efficiency of vehicles. In order to achieve optimal conversion of the pollutants, the concentrations of the three reactants, NOₓ, CO, and HC, must be in stoichiometric proportion. This is not feasible for emissions from lean burn engines, which contain low levels of carbon monoxide and hydrocarbons but high levels of nitrogen oxides. As a result, the conversion of nitrogen oxides and carbon monoxide is negatively impacted. In light of this, there is a need to develop an occlusion type catalyst with the storage-reduction property.

In the occlusion type catalytic technique, nitrogen oxides are adsorbed under lean conditions with excess oxygen and reduced to such innocuous gases as nitrogen and carbon dioxide under gas conditions. In other words, nitrogen monoxide is oxidized to nitrogen dioxide by platinum metal under lean conditions, so that they are chemically occluded as nitrogen compounds. Under conditions in which sufficient reductants, namely hydrocarbons, carbon monoxide, and hydrogen, are present, they are reduced to nitrogen and carbon dioxide by such precious metals as rhodium.

This occlusion type catalyst was spotlighted in that it can be normally applied under the lean burn condition with little modification of the conventional emission apparatuses. For the continuing development of occlusion type catalysts, a method for accurately estimating the quantity of nitrogen oxides occluded in heterogeneous catalyst should be developed. Quantitative analysis of nitrogen oxides occluded in heterogeneous catalyst is important because it enables the evaluation of the efficiency of the occlusion type catalyst as a means of meeting regulation standards on exhaust gas emissions.

Conventionally, mass spectroscopy, IR spectroscopy, microbalancing, temperature programmed desorption (TPD) using a thermal conductivity detector, and chemisorption-thermogravimetry, have been commonly employed for quantitative analysis of occluded nitrogen oxides.

Although the quantitative analysis of nitrogen oxide improves as better analytical instruments are developed, the conventional methods still require a long period of analysis time—it may take about two years to evaluate about 1,000 samples, since all the processes including pre-treatment, adsorption, purging, desorption, and calibration have to be conducted serially for each sample, requiring a large number of equipments, materials and personnel. Also, since the results obtained at each step tends to be dependent on the individual experience of the personnel involved, the results may not be reliable.

### SUMMARY OF THE INVENTION

Embodiments of the present inventor include, in parallel, a serial processes of adsorption, extraction for each sample, reduction of the extracted solutions with hydrazine, and detection and quantification of the amount of NOx occluded on the catalyst sample with a chromatic chemical sensor. Accordingly, embodiments of the present invention provides methods for quantifying an amount of nitrogen oxides occluded in heterogeneous catalysts comprising the steps of adsorption, extraction, reduction and quantification of multiple samples simultaneously.

Embodiments of the present invention relate to a method of quantifying the amount of nitrogen oxides occluded in heterogeneous catalysts, more specifically to a method of performing quantitative analysis of nitrogen oxides occluded in heterogeneous catalysts, comprising the following steps: adsorption by multiple samples using a batch adsorption apparatus, extraction of the adsorbed samples using a solvent, reduction of nitrogen oxides to nitrite ion using hydrazine, and detection and quantification by coloring using a modified Griess reagent. Each step is performed in parallel for multiple samples, thereby saving time and cost as compared to the conventional method wherein the process of serial adsorption, extraction, reduction and quantification has to be performed repeatedly to analyze the amount of NO adsorbed on the catalyst samples. Embodiments of the invention enable efficient and reliable evaluation of nitrogen oxides adsorbed onto catalyst samples, which are commonly employed in automobiles for post-treatment of exhaust gases.

One exemplary embodiment of the present invention includes by a high-throughput process comprising the adsorption of NOx by multiple samples using a batch adsorption apparatus, extraction of NOx from the samples using a solvent, reduction of the nitrogen oxides contained in the extract to nitrite ions using hydrazine, and measurement of the strength of the signal indicating the existence of NOx in the sample by colorization with a modified Griess reagent.

### BRIEF DESCRIPTION OF THE DRAWINGS

The aforementioned aspects and other features of the present invention will be explained in the following detailed description, taken in conjunction with the accompanying drawings

FIG. 1 shows the reaction scheme of each adsorption, extraction, reduction and colorization step according to the method of the present invention.

FIG. 2 shows an embodiment of a batch adsorption apparatus according to the present invention, where F stands for filter, M for gas mass flow controller, MC for mixing chamber, 3WV for three-way valve, T for thermocouple, P for pressure gauge and MS for mass spectrometer.

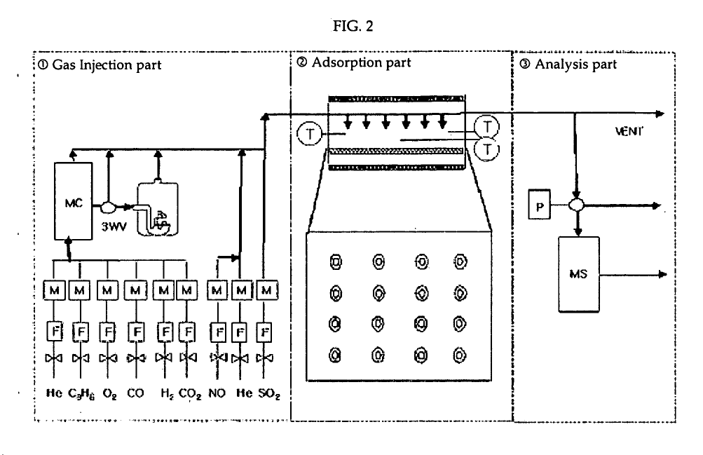

FIG. 3 shows an apparatus used for batch extraction and reduction according to the present invention.

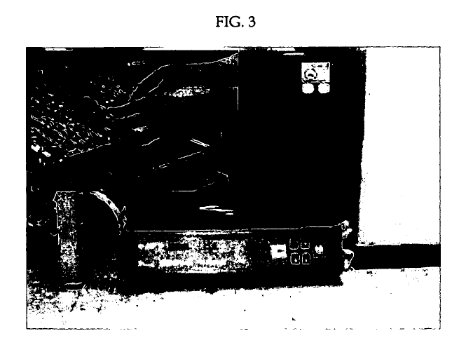

FIG. 4 shows a quantitative analysis result according to the present invention, in which the samples were colorized using a chemical sensor after having been adsorbed and extracted for 6 hours, wherein A) is Ald I, B) is AldII, C) is NaY, D) is 20% Ba/AldII, E) is 1% Pt/Ald II, F) is 1% Pt/5% Ba/Ald II, G) is 10% Ca/Ald II and H) is 1% Pt/20% Ba/Ald II.

FIG. 5 shows a quantitative analysis according to the conventional TPD, in which nitrogen oxides were desorbed from 8 samples for 80 hours, wherein A) to H) are the same as above.

FIG. 6 is a graph showing the correlation of the results of the TPD and the quantitative analysis according to the present invention.

FIG. 7 shows the quantitative analysis according to the conventional TPD, in which water, nitrogen monoxide and nitrogen dioxide were analyzed for 10 hours.

### DETAILED DESCRIPTION OF PREFERRED EMBODIMENT

The present invention relates to a method of quantifying the nitrogen oxides occluded in heterogeneous catalysts, more specifically to a method of performing quantitative analysis of the amount of nitrogen oxides occluded in heterogeneous catalysts. This process preferably comprises the following steps occurring in parallel for the various samples being tested: adsorption of NOx onto the multiple catalyst samples using a batch adsorption apparatus, extraction of the occluded samples using a solvent, reduction of nitrogen oxides contained in the extract to nitrite ions using hydrazine, and detection and quantification of the amount of NOx occluded in the catalyst samples by coloring using a modified Griess reagent.

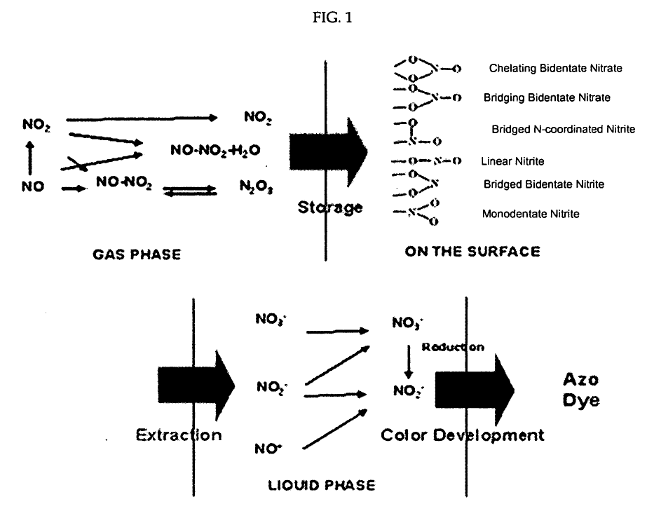

Methods of the invention reduces the time and costs needed for sample analysis as compared to the conventional method, wherein the process of serial adsorption, purge, desorption, and quantification is conducted repeatedly to quantify multiple samples. As seen in FIG. 1, nitrogen oxides are adsorbed, extracted, reduced to nitrite ions and colored. The batch adsorption apparatus used in the present invention is not particularly limited to any shape or structure.

In a gas feed step, it is preferable to inject gases along with water which is commonly contained in the exhaust stream comprising NOx. The flow rate of gases is controlled by a mass flow controller. The feed gas composition is mixed with the aid of a mixing chamber. Gas that reacts with water or is soluble in water is directly injected into the adsorption apparatus without passing through the chamber.

In an adsorption step, temperature and gas composition is preferably maintained uniformly, and multiple catalysts can be adsorbed at once. In a preferred embodiment, multiple thermocouples are used for temperature monitoring.

After the adsorption step, the change in gas composition and pressure is measured by an on-line mass spectrometer and pressure gauge, respectively.

After batch adsorption, multiple samples are extracted at once using a solvent. Any solvent commonly employed for extraction of nitrogen oxides can be used. For example, water, an alkaline aqueous solution, or an acidic aqueous solution are some of the solvents that can be used for extraction.

A batch extraction apparatus may be used to treat multiple samples simultaneously. In a preferred embodiment, the apparatus has a means of mounting multiple samples and a means of shaking the samples to achieve proper mixing of samples and solvent. The batch extraction apparatus is not limited particularly in shape or structure.

Following batch adsorption and extraction, nitrogen oxides are reduced to nitrite ions (NO₂—) using hydrazine. Reaction of hydrazine and nitrates is well known to one skilled in the area of chemistry. Griess reagent, which was designed by P. Griess, is widely used for detecting and quantifying nitrous acid and nitrite ion. In the present invention, a modified Griess reagent is used. The modified Griess reagent is a kind of chromatic chemical sensor and is not particularly limited to any one reagent for the purpose of this invention.

In a preferred embodiment, a reagent prepared by mixing a 5% phosphate solution containing sulphanyl amide and N-(1-naphtyl)ethylenediamine dihydrochloride is used. Contents of sulphanyl amide and N-(1-naphtyl)ethylenediamine dihydrochloride may be selected from what is commonly adopted in the art for coloring.

Once reaction with the modified Griess reagent occurs, the nitrite ion containing sample turns from colorless to pink. The more intense the color, the more nitrite ions are present. By measuring the absorption of the colorized reagent, the quantity of nitrogen oxides contained therein can be determined. With this simple and parallel quantitative analysis, it is possible to quickly analyze multiple samples.

Hereinafter, the present invention is described in more detail through examples. However, the following examples are provided only for illustrative purposes and not to be construed as limitations on the scope of invention.

### EXAMPLES 1-8

Samples 20% Ba/Ald II, 1% Pt/Ald II, 1% Pt/5% Ba/Ald II, 10% Ca/Ald II, 1% Pt/20% Ba/Ald II and NaY were prepared by impregnation by adding Ba, Ca and Pt to alumina Ald II and Ald II purchased from Aldrich.

Quantity nitrogen oxides was determined by the quantitative analysis method of the present invention. Weight of each sample was 20 mg. Following batch adsorption and extraction, NO₃— was reduced to NO₂— and quantitative analysis was performed by coloring the a chromatic chemical sensor.

All the samples were extracted at once. About 15 mL of neutral, acidic or alkaline aqueous solution was added to each sample. Shaking was performed for 5 minutes. One mL CuSO₄ solution (750 μg, CuSO₄ 5H₂O/20 mL H₂O), 1 mL sulfur-hydrazine solution (45 mg, N₂H₄ H₂SO₄/20 mL H₂O) and 2 mL sodium hydroxide solution (1 N NaOH) were added per 1 mL of eluted solvent.

Reduction was performed at 37° C. for 10 minutes. Then, a chromatic chemical sensor was added to colorize it. For the coloring reagent, 0.025 g of N-(1-naphtyl)ethylenediamine dihydrochloride dissolved in 5% phosphate solution (5 mL phosphate, 15 mL water) containing 0.3125 g of sulphanyl amide was used.

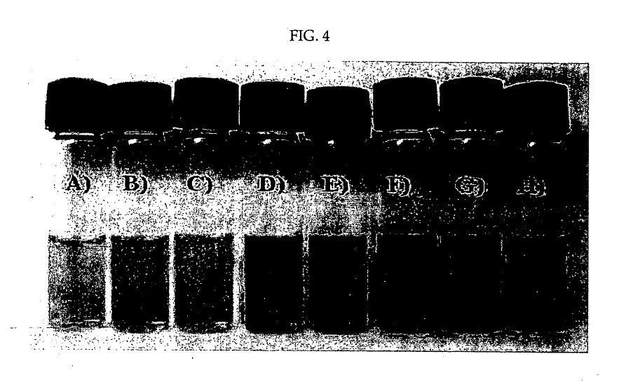

The coloring result is shown in FIG. 4. The absorption in the visible region (at 540 nm) was measured in Test Example.

### COMPARATIVE EXAMPLES 1-8

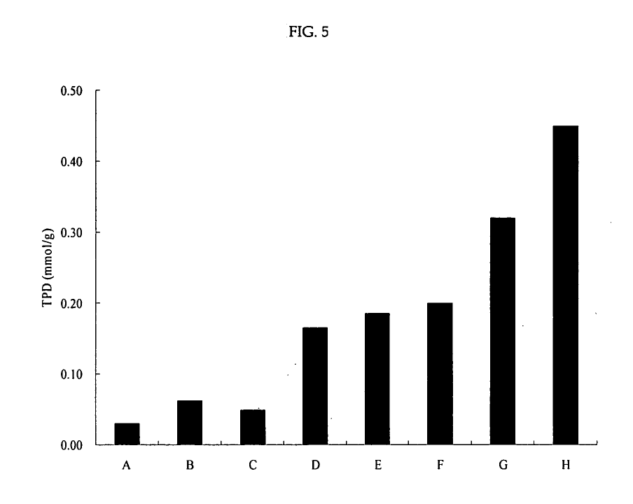

Quantity of nitrogen oxides was measured in the same manner as the Examples above using the conventional TPD method. The results are shown in FIG. 5. The analysis of 8 samples took 80 hours, about 10 hours per sample. The experiment was carried out by a skilled operator who was familiar with TPD and had at least 5 years of research experience.

### TEST EXAMPLE

Results of Examples and Comparative Examples were compared in order to confirm the advantage of the quantitative analysis using a chromatic chemical sensor according to the present invention.

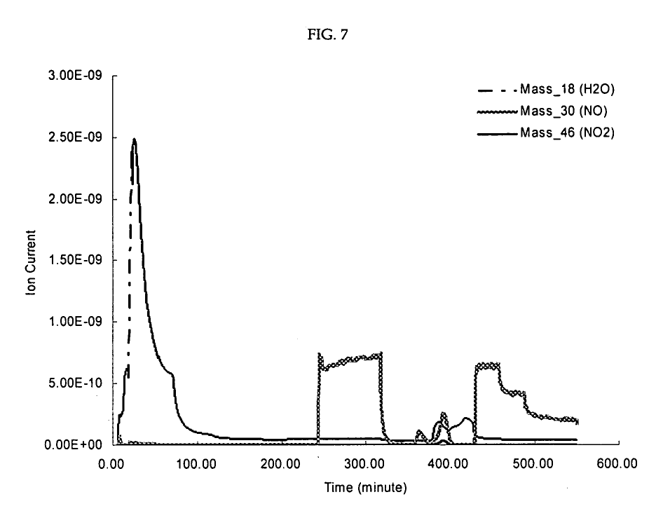

The measurement process by TPD is shown in FIG. 7. As described in Comparative Examples 1-8, the conventional TPD method took about 10 hours per sample, which adds up to about 80 hours for evaluating the nitric oxide content in 8 samples. In contrast, the method of the present inventions took only about 6 hours.

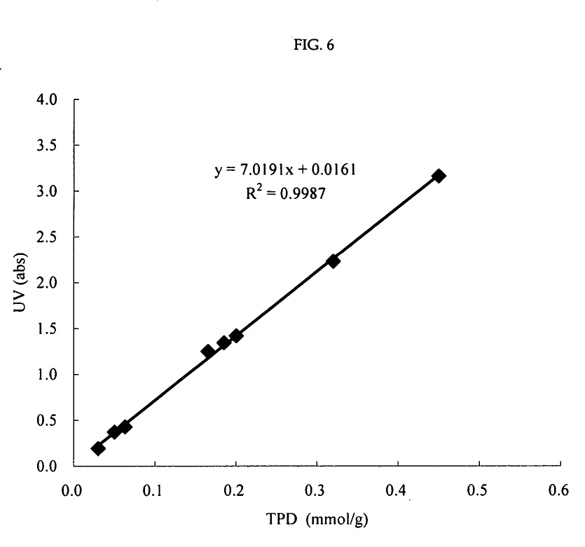

As shown in FIG. 5 and FIG. 6, the results from the method of analysis of the present invention are consistent with those of the conventional method. For quantitative comparison, adsorption amount of NOₓ determined by TPD and the absorptions (at 540 nm) of colorized samples were shown in FIG. 6. X and y had the following relationship: y=7.019x+0.0161 (R²=0.9987).

As shown in FIG. 6, a linear correlation was confirmed. Consequently, the present invention enables accurate analysis in a short period of time. Given a greater number of samples, the projected amount of time saved would be even more significant.

### INDUSTRIAL APPLICABILITY

As described above, the quantitative analysis method of the quantity of nitrogen oxides occluded in heterogeneous catalysts of the present invention is an effective high-throughput screening method of adsorption, extraction, reduction and quantification of NOx adsorbed onto multiple samples. The method of the present invention not only significantly reduces the time required for the conventional serial quantification method, but also enables fast and reliable evaluation of performance and characteristics of nitrogen oxide adsorbing catalysts currently used for post-treatment of automobile exhaust gases.

Not only does the present invention require less personnel demand, time and resources, it enables environment-friendly resource utilization by preventing emission of numerous pollutants, offers entrepreneurs an opportunity to invest in atmospheric environmental catalyst development and systematic and fast access to new techniques for industries, universities, and research institutes. This synergic effect will play a part in developing a new-generation low-pollution, environment-friendly automobiles to meet the stringent international environmental regulations.

While the foregoing description represents various embodiments of the present invention, it will be appreciated that the foregoing description should not be deemed limiting since additions, variations, modifications and substitutions may be made without departing from the spirit and scope of the present invention. It will be clear to one of skill in the art that the present invention may be embodied in other forms, structures, arrangements, and proportions and may use other elements, materials and components. The present disclosed embodiments are, therefore, to be considered in all respects as illustrative and not restrictive, the scope of the invention being indicated by the appended claims and not limited to the foregoing description.

What is to be claimed is:

1. A method of simultaneously quantifying the amount of nitrogen oxides occluded in multiple heterogeneous catalysts, comprising:
(a) adsorbing nitrogen oxides by multiple samples using a parallel adsorption apparatus;
(b) extracting nitrogen oxides from multiple samples using a solvent;
(c) reducing nitrogen oxides to nitrite ion in multiple samples using hydrazine;
(d) colorizing the solutions extracted from multiple samples with a modified Griess reagent; and
(e) measuring the absorption of the resulting solutions.

2. The method of claim 1, wherein said solvent is water.

3. The method of claim 1, wherein said solvent is an alkaline aqueous solution.

4. The method of claim 1, wherein said solvent is an acidic aqueous solution.

5. The method of claim 1, wherein said modified Griess reagent is a mixture of 5% phosphate solution containing sulphanyl amide and N-(1-naphtyl)ethylenediamine dihydrochloride.

6. A method of simultaneously quantifying the amount of nitrogen oxides occluded in multiple heterogeneous catalysts, comprising:
(a) adsorbing nitrogen oxides by multiple samples;
(b) extracting nitrogen oxides from multiple samples using a solvent;
(c) reducing nitrogen oxides to nitrite ion in multiple samples using hydrazine;
(d) colorizing the solutions extracted from multiple samples with a modified Griess reagent; and
(e) measuring the absorption of the resulting solutions.

7. The method of claim 1, wherein said solvent is water.

8. The method of claim 1, wherein said solvent is an alkaline aqueous solution.

9. The method of claim 1, wherein said solvent is an acidic aqueous solution.

10. The method of claim 1, wherein said modified Griess reagent is a mixture of 5% phosphate solution containing sulphanyl amide and N-(1-naphtyl)ethylenediamine dihydrochloride.

11. A kit comprising in a single container materials for use in quantifying the amount of nitrogen oxides occluded in a heterogeneous catalyst, comprising:
(a) a batch extraction apparatus;
(b) hydrazine; and
(c) a modified Griess reagent.

12. The kit of claim 11 wherein the modified Griess reagent is a mixture of 5% phosphate solution containing sulphanyl amide and N-(1-naphtyl)ethylenediamine dihydrochloride.

13. The kit of claim 11 further comprising a parallel absorption apparatus.

14. The kit of claim 11 further comprising a solvent for extraction of nitrogen oxides.
